(19) United States
(12) Patent Application Publication
(10) Pub. No.: US 2021/0169776 A1
(43) Pub. Date: Jun. 10, 2021

(54) COSMETIC COMPOSITION WITH A METALLIC EFFECT

(71) Applicant: CHANEL PARFUMS BEAUTE, NEUILLY SUR SEINE (FR)

(72) Inventor: Aurélie BONNEFOY, PANTIN CEDEX (FR)

(73) Assignee: CHANEL PARFUMS BEAUTE, NEUILLY SUR SEINE (FR)

(21) Appl. No.: 16/952,423

(22) Filed: Nov. 19, 2020

(30) Foreign Application Priority Data
Dec. 6, 2019 (FR) 19 13897

(51) Int. Cl. A61K 8/98; A61K 8/36; A61K 8/34; A61K 8/06; A61K 8/89; A61K 8/31; A61Q 1/04; A61Q 1/10

(52) U.S. Cl. CPC A61K 8/987; A61K 8/36; A61K 8/345; A61K 8/062; A61K 2800/436; A61K 8/31; A61Q 1/04; A61Q 1/10; A61K 2800/805; A61K 8/89

(57) ABSTRACT

A cosmetic composition including, in a continuous aqueous phase, at least one colouring agent chosen from hydrophobic treated nacres, at least one hydrophilic gelling agent and water. Also, a method for preparing such a cosmetic composition, to a method for makeup of keratin materials using same, and to the use of this cosmetic composition in order to obtain an aqueous metallic effect or “mirror effect” on the keratin materials.

### FIELD

The present invention relates to a cosmetic composition comprising, in a continuous aqueous phase, at least one colouring agent chosen from hydrophobic treated nacres, at least one hydrophilic gelling agent and water. The invention also relates to a method for preparing such a cosmetic composition, to a method for makeup of keratin materials using same, and to the use of this cosmetic composition in order to obtain an aqueous metallic effect or “mirror effect” on the keratin materials.

### BACKGROUND

The makeup or treatment products for keratin materials, such as foundations, blushes or eye shadows, generally have the function of providing colour, mattness or even providing coverage.

In general, these makeup products mainly consist of a continuous oily phase in which the quantity of fillers and colouring agents is modulated in order to procure the desired makeup effects: colour, covering, mattifying, etc.

Furthermore, metallised, iridescent or spangled effects are highly sought after for makeup products. In order to obtain such colour effects, it is known to use pigments with an optical effect, such as nacres or other interference pigments.

In order to improve their dispersion in products with a continuous oily phase, it is known to treat the surface of the colouring agents by means of a hydrophobic coating, which guarantees the most homogeneous possible colour and optical effect, and makes it possible to avoid, in particular, highlight areas.

Although the colour effects thus obtained are intense with saturated colours and satin metallic effects, these formulations in a continuous oily phase often provide a sensation of heaviness as well as a sticky effect on application that is unpleasant for the user.

Formulations having a continuous aqueous phase, are, for their part, very popular for their fresh, light and non-fatty sensory appearance. They are most often used for care products. Indeed, coloured products obtained from formulations having a continuous aqueous phase are generally insufficiently covering for makeup products. The colours lack intensity and do not exhibit a good retention over time. In addition, it is not possible to obtain special colour effects, such as an aqueous metallic effect or “mirror effect” using such galenics.

The special colouring agents, such as pigments or nacres, do not disperse homogeneously in the aqueous phase, and form aggregates which can pose stability problems. As a result, very few makeup products currently on the market have a continuous aqueous phase.

However, the search continues for fresh and comfortable cosmetic makeup compositions having an intense colour effect with saturated colours, such as an aqueous metallic effect or “mirror effect” as well as a good retention of said metallic effect over time.

The applicant has unexpectedly discovered that a composition having such properties, which at first glance are not reconcilable, can be obtained using hydrophobic treated nacres in a continuous hydrophilic phase.

### SUMMARY

The compositions according to the invention make it possible to obtain a colour effect that is an aqueous metallic or “mirror” effect, while providing a novel, long-retention, fresh and comfortable texture.

According to a first aspect, the invention relates to a cosmetic composition comprising, in a continuous aqueous phase:

at least one colouring agent chosen from hydrophobic treated nacres;
at least one hydrophilic gelling agent;
water.

According to a second aspect, the invention also relates to a method for preparing such a composition, comprising:

mixing the hydrophilic gelling agent with water, and optionally with the hydrocarbon and/or silicone emulsifiers, the humectants and the water-soluble solvents;
optionally adding film-forming agents, silicone and/or hydrocarbon emulsifiers and additives;
adding hydrophobic treated nacres and optionally hydrophobic treated pigments;
optionally adjusting the pH.

According to a third aspect, the invention also relates to a method for making up keratin materials, in particular the skin or the lips, consisting of applying on said keratin materials, in particular the skin of the lips, a composition as described above.

Finally, the invention relates to the cosmetic use of a composition as described above in order to form on the keratin materials, in particular the skin of the lips, a coloured makeup with aqueous metallic effect or “mirror effect”.

Thus, the present invention relates to a cosmetic composition comprising, in a continuous aqueous phase,

at least one colouring agent chosen from hydrophobic treated nacres;
at least one hydrophilic gelling agent; and
water.

### DETAILED DESCRIPTION

### Galenic

The composition according to the invention has a continuous aqueous phase. It is preferably in the form of an aqueous gel or an oil-in-water emulsion.

### Hydrophobic Treated Nacres

The hydrophobic treated nacres are present in the continuous aqueous phase of the composition according to the invention in a content ranging from 16 to 35% by weight, preferably from 20 to 33% by weight, and yet more preferably the content of hydrophobic treated nacres is approximately 25% by weight with respect to the total weight of the composition.

The presence of these nacres present in the continuous aqueous phase advantageously allows a novel colour effect of the aqueous metallic effect type to be obtained.

This metallic effect is obtained for hydrophobic treated nacre contents ranging from 16 to 35%. Below these contents, the desired metallic colour effect is not sufficiently visible. Above, it is too pronounced and becomes too shiny.

The nacres can be chosen from those conventionally present in makeup products, such as mica/titanium dioxide based nacres (for example, DK PEARL SILVER 310 marketed by DAITO KASEI EUROPE), mica/titanium oxide/iron oxide based nacres (such as DK PEARL ORANGE GOLD, DK PEARL DARK RED or DK PEARL GOLD marketed by DAITO KASEI EUROPE), mica/iron oxide based nacres (for example DK PEAR BRONZE marketed by DAITO KASEI EUROPE) mica/silica/titanium dioxide based nacres and synthetic fluorphlogopite/titanium dioxide based nacres (SUNSHINE® from MAPRECOS), calcium sodium borosilicate/titanium dioxide (REFLECKS® from ENGELHARD) or calcium aluminium borosilicate/silica/titanium dioxide (RONASTAR® from MERCK).

According to a preferred embodiment, the nacres are chosen from mica/titanium dioxide based nacres, mica/titanium oxide/iron oxide based nacres, mica/iron oxide based nacres, and the mixtures thereof.

According to an embodiment, the nacres are hydrophobically treated nacres.

According to an embodiment, the hydrophobic treatment is a treatment with metallic soap.

Indeed, the nacres are at least partially, preferably totally, treated at the surface by a metallic soap.

Typically, the metallic soap is a fatty acid soap having 12 to 22 carbon atoms, and in particular 12 to 18 carbon atoms.

The metal of the metallic soap is in turn preferably chosen from zinc and magnesium.

Hence, according to a preferred embodiment, the metallic soap is chosen from zinc laurate, magnesium stearate, magnesium myristate, zinc stearate and the mixtures thereof.

Preferably, the metallic soap is magnesium stearate.

### Hydrophilic Gelling Agent

The continuous aqueous phase of the composition according to the invention comprises at least one hydrophilic gelling agent.

Gelling agent means a compound which, in the presence of a solvent, creates more or less strong inter-macromolecular bonds, thus inducing a three-dimensional network which freezes said solvent.

The gelling agent is an aqueous phase gelling agent which advantageously allows the hydrophobically treated nacres to disperse in the aqueous phase as well as keeping them in suspension.

The gelling agent may be chosen from polysaccharides, polyacrylates, polymethacrylates and the derivatives thereof.

The polysaccharides include:

the exudates of microorganisms such as xanthan gum and the derivatives thereof such as the products sold under the tradename Rhéosan by Rhodia Chimie, the product sold under the tradename KELTROL® CG-SFT (INCI: xanthan gum) by CP Kelco, or the product SAFIC CARE T XCG (INCI: Ceratonia siliqia gum & xanthan gum) from SAFIC ALCAN, gellan gum sold under the tradename Kelcogel F by NUTRASWEET-KELCO or again iota-carrageenan sold under the tradenames Seaspen PF 357 or Viscarin SD 389 by FMC, or sclerotium gum (or sclerotium rolfssii gum), produced by the bacterium Sclerotium rolfissii, available under the name Naturajel® from DIY Cosmétics or Amigel® from Alban Muller;
plant extracts such as polysaccharides of tremella fuciformis (INCI: Tremella fuciformis polysaccharide), such as the product sold under the tradename Tremoist™-TP by Nippon Fine Chemical;
algal extracts such as agar-agar, carrageenans (iota, kappa, lambda) such as the products sold under the tradename Viscarin PC 209 (INCI: Carrageenan) by FMC BioPolymer, and the derivatives thereof such as the product sold under the tradename Sucraclear HC-31 (INCI: Cellulose Gum, Carrageenan, Ceratonia Siliqua Gum, Sucrose) by ALCHEMY ingredients, alginates, in particular Na or Ca;
and the mixtures thereof

The polyacrylates include:

acrylic acid polymers such as carbomer, like that sold under the tradename Carbobol Ultrez 10 (INCI: carbomer), by Lubrizol or Gattefosse France,
cross-linked methyl acrylate and 25 EO polyoxyethylenated behenyl methacrylate polymers (INCI name: Acrylates/Beheneth-25 Methacrylate Copolymer), such as that sold under the tradename Novethix L-10 Polymer by Lubrizol Advanced Materials, Rheostyl™ 90 N from Arkema, or Aculyn™ 28 from Dow Chemical.

Preferably, the gelling agent is chosen from the polysaccharides such as the exudates of microorganisms, or plant-extract polysaccharides, acrylic acid polymers or one of the mixtures thereof.

More preferably, the gelling agent is chosen from carbomer, xanthan gum, tremella fuciformis polysaccharide, or one of the mixtures thereof.

According to an embodiment, the continuous aqueous phase of the composition according to the invention comprises a single aqueous-phase gelling agent, preferably carbomer.

According to an embodiment, the gelling agent can have a content ranging from 0.05% to 3% by weight, preferably from 0.1 to 2% by weight, and yet more preferably from 0.3 to 2% by weight, with respect to the total weight of the composition.

### Aqueous Phase

The composition according to the invention comprises an aqueous phase comprising water.

According to a particular embodiment, the composition according to the invention comprises 30 to 70% by weight water, preferably 40 to 60% by weight and, yet more preferably approximately 50% by weight water, with respect to the total weight of the composition.

The water contents of the composition according to the invention can advantageously confer an immediate fresh effect on the cosmetic composition. Once applied, the cosmetic composition according to the invention is light, without a sensation of heaviness or sticking. It is thus possible to obtain a fresh and comfortable makeup cosmetic composition.

### Water-Soluble Solvents

The composition according to the invention further comprises in the continuous aqueous phase, at least one water-soluble solvent.

Herein, “water-soluble solvent” designates a compound that is liquid at room temperature and miscible with water (miscibility in water greater than 50% by weight at 25° C. an atmospheric pressure.

The water-soluble solvents which may be used in the compositions according to the invention can be volatile.

The water-soluble solvents which can be used in the compositions according to the invention include monoalcohols having 1 to 5 carbon atoms, such as ethanol and isopropanol, the C₃-C₄ ketones and C₂-C₄ aldehydes

Hence, according to a preferred embodiment, the composition according to the invention comprises at least one monoalcohol having 1 to 5 carbon atoms, preferably ethanol.

Typically, the monoalcohol having 1 to 5 atoms is present in a content ranging from 0.1 to 10% by weight, with respect to the total weight of the composition.

The introduction of a monoalcohol having 1 to 5 carbon atoms facilitates and accelerates the drying of the composition.

### Humectants

The composition according to the invention comprises at least one humectant.

The humectants include, in particular, polyols.

Hence, the composition according to the invention further comprises a humectant chosen from the polyols.

Polyol means any organic molecule having at least two free hydroxy groups (—OH) in its structure. These polyols are preferably liquid at room temperature (25° C.).

By way of example, the polyols suitable for use in the composition can be chosen from propylene glycol, butylene glycol, pentylene glycol, pentanediol, isoprene glycol, neopentyl glycol, glycerol, polyethylene glycols (PEG) having in particular from 4 to 8 ethylene glycol and/or sorbitol units.

Preferably, the humectant is chosen from glycerol and butylene glycol.

According to a particular embodiment, the composition according to the invention comprises 0 to 20% by weight humectant, preferably 5 to 10% by weight humectant, with respect to the total weight of the composition, and in a yet more preferred manner, the composition according to the invention comprises approximately 6% by weight humectant, with respect to the total weight of the composition.

### Pigments

The cosmetic composition according to the invention further comprises, in its aqueous phase, at least one other colouring agent chosen from the hydrophobic treated pigments, the content of hydrophobic treated pigments with respect to the total weight of the composition being less than 1.5%.

Advantageously, the applicant has demonstrated that it is possible to introduce hydrophobic treated pigments, in a content less than 1.5% by weight with respect to the total weight of the composition, into the compositions in the continuous aqueous phase according to the invention, in order to reinforce the lustre and the colour effect and to prevent the colours from fading, and this without destabilising the composition. More particularly, the applicant has shown that it is possible to obtain gels of carbomer containing iron oxides in order to provide the colour without destabilising the gel.

According to an embodiment, the composition contains no hydrophobic treated pigments.

“Pigments” should be understood to mean mineral or organic, white or coloured particles, insoluble in an aqueous medium, intended for colouring and/or opacifying the composition.

The pigments may be white or coloured, mineral and/or organic.

The pigment may be an organic pigment. The organic pigment may be chosen from nitroso, nitro, azo compounds, xanthene, quinoline, anthraquinone and phthalocyanine, of the metal complex type, isoindolinone, isoindoline, quinacridone, perinone, perylene, dicetopyrrolopyrrole, thioindigo, dioxazine, triphenylmethane and quinophtalone.

The one or more organic pigments can be chosen, for example, from carmine, carbon black, aniline black, melanin, azo yellow, quinacridone, phthalocyanine blue, sorghum red, the blue pigments codified in the Colour Index under references C1 42090, 69800, 69825, 73000, 74100, 74160, the yellow pigments codified in the Colour Index under references CI 11680, 11710, 15985, 19140, 20040, 21100, 21108, 47000, 47005, the green pigments codified in the Colour Index under references CI 61565, 61570, 74260, the orange pigments codified in the Colour Index under references CI11725, 15510,45370, 71105, the red pigments codified in the Colour Index under references CI 12085, 12120, 12370, 12420, 12490, 14700, 15525, 15580, 15620, 15630, 15800, 15850, 15865, 15880, 17200, 26100, 45380, 45410, 58000, 73360, 73915, 75470, the pigments obtained by oxidative polymerisation of indole and phenolic derivatives, as described in patent FR 2 679 771.

These pigments can also be formed of composite pigments, as described in patent EP 1 184 426. These composite pigments can, in particular, be composed of particles having an inorganic core, covered at least partially by an organic pigment and at least one binder ensuring the attachment of the organic pigments on the core.

The pigments can also be a lacquer. Herein, lacquer shall mean the insolubilised dyes absorbed on the insoluble particles, the assembly thus obtained remaining insoluble during use. By way of example, the lacquers include the product known under the following name: D & C Red 7 (CI 15 850:1).

The pigment can be a mineral pigment. Mineral pigment means any pigment which responds to the Ullmann Encyclopaedia definition given in the chapter on inorganic pigment. The mineral pigments used in the present invention include zirconium or cerium oxides, as well as the oxides of zinc, iron (black, yellow or red) or chromium, manganese violet, ultramarine blue, chromium hydrate and ferric blue, titanium dioxide, metal powders such as aluminium powder and copper powder. The following mineral pigments can also be used: Ti₂O₅, Ti₃O₅, Ti₂O₃, TiO, ZrO₂ in mixture with TiCO₂, ZrO₂, Nb₂O₅, CeO₂, ZnS.

According to a preferred embodiment, the pigment is a mineral pigment such as an iron oxide, preferably a black iron oxide.

The size of the pigment used in the context of the present invention is generally between 10 nm and 10 μm, preferably between 20 nm and 5 μm, and more preferably between 30 nm and 1 μm.

According to an embodiment, the hydrophobic treatment of the pigments is a metallic soap treatment.

Indeed, the pigments are at least partially, preferably totally, treated at the surface by a metallic soap.

Typically, the metallic soap is a fatty acid soap having 12 to 22 carbon atoms, and in particular 12 to 18 carbon atoms.

The metal of the metallic soap is in turn preferably chosen from zinc and magnesium.

Hence, according to a preferred embodiment, the metallic soap is chosen from zinc laurate, magnesium stearate, magnesium myristate, zinc stearate and the mixtures thereof.

Preferably, the metallic soap is magnesium stearate.

### Fillers

The composition according to the invention may also comprise at least one filler. These fillers serve in particular to modify the rheology or the texture of the composition.

The fillers can be mineral or organic and of any shape, platelet, spherical or oblong, whatever the crystallographic form (for example laminar, cubic, hexagonal, orthorhombic, etc.). These include talc, mica, mica surface-treated with a hydrophobic agent, cellulose, cellulose surface-treated with a hydrophobic agent, silica, silica surface-treated with a hydrophobic agent, kaolin, polyamide powders (Nylon®) (Orgasol® from Atochem), poly-β-alanine and polyethylene, polymer powders of tetrafluoroethylene (Teflon®), lauroyl-lysine, starch, boron nitride, hollow polymer microspheres such as those of polyvinylidene chloride/acrylonitrile such as Expancel® (Nobel Industrie), acrylic acid copolymers (Polytrap® from DOWSIL) and microspheres of silicone resin (Tospearls® from Toshiba, for example), polyorganosiloxane elastomer particles, precipitated calcium carbonate, magnesium carbonate and bicarbonate, hydroxyapatite, hollow silica microspheres (Silica Beads® from Maprecos), glass or ceramic microcapsules, metallic soaps derived from organic carboxylic acids having 8 to 22 carbon atoms, preferably 12 to 18 carbon atoms, for example zinc, magnesium or lithium stearate, zinc laurate and magnesium myristate.

According to an embodiment, the filler is a mica that is hydrophobically treated by metallic soap, preferably a fatty acid soap having 12 to 22 carbon atoms, and in particular 12 to 18 carbon atoms. The metal of the metallic soap is preferably zinc or magnesium.

By way of illustration, this may be MICA SX marketed by DAITO KASEI EUROPE.

### Film-Forming Agent

The composition according to the invention further comprises a film-forming agent, in particular a film-forming polymer.

According to a first embodiment, the film-forming polymer may be present in the composition of the invention in the form of particles in dispersion in an aqueous phase. A film-forming polymer dispersible in an aqueous phase is generally referred to as latex or pseudolatex. Techniques for preparing these dispersions are well known to a person skilled in the art.

As film-forming polymer that is dispersible in the aqueous phase or aqueous dispersion of film-forming polymer, it is possible to use acrylic dispersions sold under the names Neocryl XK-90®, Neocryl A-1070®, Neocryl A-1090®, Neocryl BT-62®, Neocryl A-1079® and Neocryl A-523® by AVECIA-NEORESINS, Dow Latex 432® by DOW CHEMICAL, Daitosol 5000 AD® or Daitosol 5000 SJ® by DAITO KASEY KOGYO; Syntran 5760® by Interpolymer, Allianz OPT by ROHM & HAAS, the aqueous dispersions of acrylic or styrene/acrylic polymers sold under the tradename JONCRYL® by JOHNSON POLYMER or again aqueous dispersions of polyurethane sold under the names Neorez R-981® and Neorez R-974® by AVECIA-NEORESINS, Avalure UR-405®, Avalure UR-410®, Avalure UR-425®, Avalure UR-450®, Sancure 875®, Sancure 861®, Sancure 878® and Sancure 2060® by GOODRICH, Impranil 85® by BAYER, Aquamere H-1511® by HYDROMER; sulfopolyesters sold under the tradename Eastman AQ® by Eastman Chemical Products, vinyl dispersions such as Mexomère PAM® from CHLMEX and the mixtures thereof.

According to a second embodiment, the film-forming polymer may be a water-soluble polymer and may therefore be present in the continuous aqueous phase of the composition according to the invention.

According to a third embodiment, the film-forming polymer may be a polymer solubilised in a liquid fatty phase comprising oils or organic solvents, the film-forming polymer is therefore referred to as a lipophilic (liposoluble or lipodispersible) film-forming polymer.

Examples of liposoluble polymer include the vinyl ester copolymers (the vinyl group being bonded directly to the oxygen atom of the ester group and the vinyl ester having a linear or branched, saturated hydrocarbon radical with 1 to 19 carbon atoms, bonded to the carbonyl of the ester group) and at least one other monomer which may be a vinyl ester (different from the vinyl ester already present), an a-olefin (having 8 to 28 carbon atoms), an alkylvinyl ether (the alkyl group of which comprises 2 to 18 carbon atoms), or an allyl or methallyl ester (having a linear or branched, saturated hydrocarbon radical with 1 to 19 carbon atoms, bonded to the carbonyl of the ester group).

These polymers can be cross-linked using cross-linking agents which can be either vinyl type, or allylic or methallylic type, such as tetraallyloxyethane, divinylbenzene, divinyl octanedioate, divinyl dodecanedioate, and divinyl octadecanedioate.

Examples of these copolymers include the copolymers: vinyl acetate/allyl stearate, vinyl acetate/vinyl laurate, vinyl acetate/vinyl stearate, vinyl acetate/octadecene, vinyl acetate/octadecylvinyl ether, vinyl propionate/allyl laurate, vinyl propionate/vinyl laurate, vinyl stearate/octadecene-1, vinyl acetate/dodecene-1, vinyl stearate/ethylvinyl ether, vinyl propionate/cetyl vinyl ether, vinyl stearate/allyl acetate, vinyl dimethyl-2, 2 octanoate/vinyl laurate, allyl dimethyl-2, 2 pentanoate/vinyl laurate, vinyl dimethyl propionate/vinyl stearate, allyl dimethyl propionate/vinyl stearate, vinyl propionate/vinyl stearate, cross-linked with 0.2% divinyl benzene, vinyl dimethyl propionate/vinyl laurate, cross-linked with 0.2% divinyl benzene, vinyl acetate/vinyl octadecyl ether, cross-linked with 0.2% tetraallyloxyethane, vinyl acetate/allyl stearate, cross-linked with 0.2% divinyl benzene, vinyl acetate/octadecene-1 cross-linked with 0.2% divinyl benzene and allyl propionate/allyl stearate cross-linked with 0.2% divinyl benzene.

Film-forming liposoluble polymers can also include liposoluble copolymers, and in particular those resulting from copolymerisation of vinyl esters having 9 to 22 carbon atoms or alkyl acrylates or methacrylates and allyl radicals having 10 to 20 carbon atoms.

Such liposoluble copolymers can be chosen from the copolymers of vinyl polystearate, vinyl polystearate cross-linked using divinylbenzene, diallyl ether or diallyl phthalate, the copolymers of stearyl poly(meth)acrylate, vinyl polylaurate, lauryl poly(meth)acrylate, these poly(meth)acrylates being able to be cross-linked using methylene glycol dimethacrylate or tetraethylene glycol.

The liposoluble copolymers defined above are known and in particular are described in application FR-A-2232303; they can have a weight average molecular weight in the range from 2000 to 500,000 and preferably from 4000 to 200,000.

The liposoluble homopolymers are also included, and in particular those resulting from homopolymerisation of vinyl esters having 9 to 22 carbon atoms or alkyl acrylates or methacrylates and allyl radicals having 2 to 24 carbon atoms.

Examples of liposoluble homopolymers include, in particular: vinyl polylaurates and lauryl poly(meth)acrylates, these poly(meth)acrylates being able to be cross-linked using ethylene glycol dimethacrylate or tetraethylene glycol.

Film-forming liposoluble polymers that can be used in the invention also include polyalkylenes and in particular C2-C20 alkene copolymers, such as polybutene, alkylcelluloses with a C1-C8, linear or branched, saturated or unsaturated, alkyl radical such as ethylcellulose and propylcellulose, the copolymers of vinylpyrrolidone (VP) and in particular the copolymers of vinylpyrrolidone and C2-C40, preferably C3-C20, alkene. By way of example, the VP copolymer that can be used in the invention includes the copolymers VP/vinyl acetate, VP/ethyl methacrylate, butyl polyvinylpyrolidone (PVP), VP/ethyl methacrylate/methacrylic acid, VP/eicosene, VP/hexadecene, VP/triacontene, VP/styrene and VP/acrylic acid/lauryl methacrylate.

Also included are silicone resins, generally soluble or swellable in silicone oils, which are cross-linked polyorganosiloxane polymers. The nomenclature of silicone resins is known under the name “MDTQ”, the resin being described as a function of the different siloxane monomer units that it comprises, each of the letters “MDTQ” characterising a type of unit.

By way of example, commercially available polymethylsilsesquioxane resins may include those marketed by Wacker Chemie AG under reference Resin MK, such as Belsil PMS MK, and by SHIN-ETSU under references KR-220L

Siloxysilicate resins include trimethylsiloxysilicate (TMS) resins such as those marketed under reference SR1000 by General Electric or under the reference Belsil TMS 803 by Wacker Chemie AG. Also included are trimethylsiloxysilicate resins marketed in a solvent such as cyclomethicone, sold under the name KF-7312J by Shin-Etsu, DOWSIL™ RSN-0749, DOWSIL™ 593 Fluid by DOWSIL. Also included are aqueous dispersions of trimethylsiloxysilicate resins, sold under the name GRANRESIN SIW-MQIZ (INCI: Isododecane (and) Trimethylsiloxysilicate (and) Aqua (and) Propanediol (and) Decyl Glucoside) marketed by GRANT INDUSTRIES.

Also included are copolymers of silicone resins such as those cited above with polydimethylsiloxanes, such as pressure-sensitive adhesive copolymers marketed by DOWSIL under reference BIO-PSA and described in document U.S. Pat. No. 5,162,410 or again the silicon copolymers originating from the reaction of a silicone resin, such as those described above, and a diorganosiloxane as described in document WO 2004/073626.

Also included are acrylate/polytrimethylsiloxymethacrylate copolymers comprising a dendrimer carbosiloxane structure grafted onto a vinyl backbone commercially available under references DOWSIL FA 4002 ID or DOWSIL FA 4001 CM, or DOWSIL FA 4103 (aqueous dispersion).

Finally, it is possible to use silicone polyamides of the polyorganosiloxane type as described in documents U.S. Pat. Nos. 5,874,069, 5,919,441, 6,051,216 and 5,981,680.

According to a preferred embodiment, the film-forming polymer is chosen from polymers of natural origin, optionally modified, preferably from polymers extracted from the fruit of Caesalpinia spinosa and/or the seaweed Kappaphycus alvarezii (such as the product Filmexel® marketed by Silab).

According to a preferred embodiment, the film-forming agent is chosen from a film-forming polymer dispersible in an aqueous phase, preferably, an aqueous dispersion of trimethylsiloxysilicate resins, sold under the name GRANRESIN SIW-MQIZ (INCI: Isododecane (and) Trimethylsiloxysilicate (and) Aqua (and) Propanediol (and) Decyl Glucoside) marketed by GRANT INDUSTRIES, a liposoluble film-forming polymer such as the trimehylsiloxysilicate as marketed under the reference Belsil TMS 803 by Wacker Chemie AG dispersed in isododecane, acrylate/polytrimethylsiloxymethacrylate copolymers comprising a dendrimer carbosiloxane structure grafted on a vinyl backbone commercially available under references DOWSIL FA 4002 ID, and the mixtures thereof.

According to an embodiment, the film-forming agent is present in the composition according to the invention with a content between 5 and 20% by weight, preferably between 5 and 15% by weight, and yet more preferably between 10 and 15% by weight, with respect to the total weight of the composition.

Thus, the cosmetic compositions according to the invention, in addition to the metallic effect as well as the freshness and comfort of application, also provide a long retention after application thanks to the film-forming agent used.

Long retention means a cosmetic composition which does not fade, or fades very slightly, which does not lose, or very slightly loses, homogeneity, does not migrate and does not defuse after application for a period of at least 3 hours 30 minutes, preferably at least 7 hours 30 minutes

### Oily Phase

The composition according to the invention may comprise at least one oil and at least one emulsifier, said composition being an oil-in-water emulsion.

According to an embodiment, the oil will be chosen from volatile oils and/or non-volatile oils, and the mixtures thereof.

According to a preferred embodiment, the oil is a volatile oil.

In the sense of the invention, “volatile oil” means an oil capable of evaporating on contact with keratin fibres in less than one hour, at room temperature and atmospheric pressure. The one or more volatile organic solvents and the volatile oils of the invention are volatile organic solvents and cosmetic oils that are liquid at room temperature, having a non-zero vapour pressure at room temperature and atmospheric pressure ranging in particular from 0.13 Pa to 40,000 Pa (10⁻³ to 300 mm Hg), in particular ranging from 1.3 Pa to 13,000 Pa (0.01 to 100 mm Hg), and more particularly ranging from 1.3 Pa to 1300 Pa (0.01 to 10 mm Hg).

According to an embodiment, the volatile oil is chosen from volatile hydrocarbon oils, volatile linear alkanes, and volatile silicone oils.

The volatile oil can be a hydrocarbon. The volatile hydrocarbon oil can be chosen from hydrocarbon oils having 7 to 16 carbon atoms. Volatile hydrocarbon oil having 7 to 16 carbon atoms includes, in particular, C8-C16 branched alkanes such as the C8-C16 isoalkanes (also called isoparaffins), isododecane, isodecane, isohexadecane and, for example, the oils sold under the tradenames Isopars or Permetyls, C8-C16 branched esters such as isohexyl neopentanoate, and the mixtures thereof. The volatile hydrocarbon oil having 8 to 16 carbon atoms is preferably chosen from isododecane, isodecane, isohexadecane and the mixtures thereof, and is in particular isododecane.

The volatile oil may be a volatile linear alkane. According to an embodiment, an alkane suitable for the invention may be a volatile linear alkane comprising 7 to 14 carbon atoms. Such a volatile linear alkane may advantageously be of plant origin. Examples of alkanes suitable for the invention include the alkanes described in the patent applications from Cognis, WO 2007/1068371 or WO2008/155059 (distinct alkane mixtures differing by at least one carbon). These alkanes are obtained from fatty alcohols, themselves obtained from coconut or palm oil. By way of example, linear alkanes suitable for the invention may include n-heptane (C7), n-octane (C8), n-nonane (C9), n-decane (010), n-undecane (C11), n-dodecane (C12), n-tridecane (C13), n-tetradecane (C14), and the mixtures thereof. According to a particular embodiment, the volatile linear alkane is chosen from n-nonane, n-undecane, n-dodecane, n-tridecane, n-tetradecane, and the mixtures thereof. A preferred mode includes the mixtures of n-undecane (C11) and n-tridecane (C13) obtained in examples 1 and 2 of application WO2008/15505 from Cognis. The mixture of n-undecane (C11) and n-tridecane (C13) marketed by BASF under the name CETIOL ULTIMATE is also included. Also included are n-dodecane (C12) and n-tetradecane (C14) sold by Sasol respectively under references PARAFOL 12-97 and PARAFOL 14-97, as well as the mixtures thereof. It is possible to use the volatile linear alkane alone or preferably a mixture of at least two separate volatile linear alkanes, differing from each other by a carbon number n of at least 1, in particular differing from each other by a carbon number of 1 or 2.

The volatile oil may be a volatile silicon oil such as the cyclic polysiloxanes, linear polysiloxanes and the mixtures thereof. The linear volatile polysiloxanes include hexamethyldisiloxane, octamethyltrisiloxane, decamethyltetrasiloxane, tetradecamethylhexasiloxane and hexadecamethylheptasiloxane. Volatile cyclic polysiloxanes include hexamethylcyclotrisiloxane, octamethylcylotetrasiloxane, decamethylcyclopentasiloxane, dodecamethylcyclohexasiloxane, cyclopentasiloxane.

According to an embodiment, the volatile oil is present in a content ranging from 0 to 20% by weight, preferably 3 to 10% by weight, with respect to the total weight of the composition.

“Non-volatile oil” means an oil remaining on the keratin fibres at room temperature and atmospheric pressure for at least several hours and having, in particular, a vapour pressure less than 10⁻³ mm Hg (0.13 Pa).

The non-volatile oils can, in particular, be chosen from non-volatile fluorinated hydrocarbon oils/or silicone oils.

Non-volatile hydrocarbon oils may include, in particular:

hydrocarbon oils of animal origin,
hydrocarbon oils of plant origin such as C4 to C36, preferably C11-C21, linear alkanes, such as phytosqualane or Emogreen L15 from SEPPIC (C15-19 alkane), or again such as the phytostearyl esters, such as phytostearyl oleate, phytostearyl isostearate and lauroyl/octyldodecyl/phytostearyl glutamate (AJINOMOTO, ELDEW PS203), triglycerides formed of fatty acid esters and glycerol, in particular, for which the fatty acids can have chain links varying from C4 to C36, and, in particular, form C18 to C36; these oils can be linear or branched, saturated or unsaturated; these oils can in particular be heptanoic or optanoic triglycerides, oils from shea, alfalfa, poppyseed, red kuri squash, millet, barley, quinoa, rye, candlenut, passion flower, shea butter, aloe oil, sweet almond oil, peach almond oil, peanut oil, argan oil, avocado oil, baobab oil, borage oil, broccoli oil, calendula oil, camelina oil, carrot oil, safflower oil, hemp oil, rapeseed oil, cottonseed oil, coconut oil, pumpkin seed oil, wheat germ oil, jojoba oil, lily oil, macadamia oil, corn oil, meadowfoam oil, St. John's wort oil, monoi oil, hazelnut oil, apricot kernel oil, walnut oil, olive oil, evening primrose oil, palm oil, blackcurrant seed oil, kiwi seed oil, grape seed oil, pistachio oil, red kuri squash oil, pumpkin oil, quinoa oil, rosehip oil, sesame oil, soybean oil, sunflower oil, castor oil, and watermelon oil, and the mixtures thereof, or even caprylic/capric acid triglycerides, such as those sold by STEARINERIES DUBOIS or those sold under the names MIGLYOL 810®, 812® and 818® by DYNAMIT NOBEL,
synthetic esters having 10 to 40 carbon atoms;
synthetic esters, such as oils of formula R1 COOR2, wherein R1 represents a residue of a linear or branched fatty acid having 1 to 40 carbon atoms and R2 represents a hydrocarbon chain, in particular branched, containing 1 to 40 carbon atoms under the condition that R1+R2 is ≥10. The esters can be chosen, in particular, from alcohol esters and fatty acid esters, such as for example cetostearyl octanoate, isopropylic alcohol esters, such as isopropyl myristate, isopropyl palmitate, ethyl palmitate, 2-ethyl-hexyl palmitate, isopropyl stearate or isostearate, isostearyl isostearate, octyl stearate, hydroxyl esters, such as isostearyl lactate, octyl hydroxystearate, diisopropyl adipate, heptanoates, and in particular isostearyl heptanoate, alcohol or polyalcohol octanoates, decanoates or ricinoleates, such as propylene glycol dioctanoate, cetyl octanoate, tridecyl octanoate, ethyl 2-hexyl 4-diheptanoate and palmitate, alkyl benzoate, polyethylene glycol diheptanoate, propylene glycol dietyl 2-hexanoate and the mixtures thereof, C12-C15 alcohol benzoates, hexyl laurate, neopentanoic acid esters, such as isodecyl neopentanoate, isotridecyl neopentanoate, isostearyl neopentanoate, octyldocecyl neopentanoate, isononanoic acid esters, such as isononyl isononanoate, isotridecyl isononanoate, octyl isononanoate, hydroxyl esters such as isostearyl lactate, di-isostearyl malate; ethyl-hexyl polyol esters and pentaerythritol esters, such as dipentaerythritol tetrahydroxystearate/tetraisostearate, ethyl-hexyl dimer diol esters and dimer diacid esters, such as Lusplan DD-DA5® and Lusplan DD-DA7®, marketed by NIPPON FINE CHEMICAL and described in application US 2004-175338,
the dimer diol and dimer diacid copolymers and the esters thereof, such as dimer dilinoleyl diol/dimer dilinoleic copolymers and the esters thereof, such as for example Plandool-G,
polyol and dimer diacid copolymers, and the esters thereof, such as Hailuscent ISDA,
fatty alcohols that are liquid at room temperature with branched and/or unsaturated carbon chains having 12 to 26 carbon atoms, such as 2-octyldodecanol, isostearylic alcohol, oleic alcohol, 2-hexyldecanol, 2-blatyloctanol and 2-undecylpentadecanol,
C₁₂-C₂₂ higher fatty acids, such as oleic acid, linoleic acid, and the mixtures thereof,
dialkyl carbonates, the 2 alkyl chains being identical or different, such as dicaprylyl carbonate marketed under the name CETIOL CC®, by COGNIS,
high molar mass oils having, in particular, a molar mass ranging from approximately 400 to approximately 10,000 g/mol, in particular from approximately 650 to approximately 10,000 g/mol, in particular from approximately 750 to approximately 7500 g/mol, and more particularly varying from approximately 1000 to approximately 5000 g/mol,
silicone oils, such as phenyl silicones, such as BELSIL PDM 1000 from WACIER (MM=9000 g/mol). Other non-volatile silicone oils which can be used in the composition according to the invention can be non-volatile polydimethylsiloxanes (PDMS), the PDMS having alkyl or alkoxy groups that are pendant and/or at the silicon chain ends, groups each having 2 to 24 carbon atoms, phenyl silicones, such as phenyl trimethicones, phenyl dimethicones, phenol trimethylsiloxy diphenylsiloxanes, diphenyl dimethicones, diphenyl methyldiphenyl trisiloxanes, and 2-phenylethyl trimethylsiloxysilicates, dimethicones or phenyltrimethicone of viscosity less than or equal to 100 cSt, and the mixtures thereof,
the fluorinated oils usable in the invention are, in particular, fluorosilicone oils, fluorinated polyethers and fluorinated silicones as described in document EP-A-847752.

According to an embodiment, the cosmetic composition according to the invention comprises at least 5% by weight non-volatile oils with respect to the total weight of the composition, and preferably the composition is free of non-volatile oils.

Advantageously, a non-volatile oil content less than 5% by weight with respect to the total weight of the composition and preferably a composition free of non-volatile oils enables a composition having improved retention to be obtained.

### Emulsifier

The emulsifier can be chosen from hydrocarbon emulsifiers, silicone emulsifiers, or the mixtures thereof.

The hydrocarbon emulsifiers may include:

fatty acid esters (in particular C8-C24, and preferably C16-C22 acids) and oxyethylenated and/or oxypropylenated sorbitol esters (able to have 1 to 150 oxyethylenated and/or oxypropylenated groups), such as polysorbate 20, in particular sold under the tradename Tween 20® by CRODA, polysorbate 60, in particular sold under the tradename Tween 60® by CRODA.

Among the silicone emulsifiers, it is possible to use those named INCI PEG-10 Dimethicone, such as those marketed by DKSH France or by Shin-Etsu Silicones, under the name KF-6017.

According to a preferred embodiment, the emulsifier is chosen from PEG-10 Dimethicone, polysorbate 20 or polysorbate 60, or the mixtures thereof.

According to an embodiment, the emulsifier is present in a content ranging from 0.05% to 3% by weight, preferably from 0.1 to 2% by weight with respect to the total weight of the composition, and more preferably from 0.3 to 1% by weight with respect to the total weight of the composition.

### Aqueous Metallic Colour Effect

The composition according to the invention makes it possible to obtain an aqueous metallic colour effect or “mirror effect” in compositions having a continuous aqueous phase.

Aqueous metallic colour effect means an optical effect giving the colour a shiny homogeneous effect, reflecting the light, like a “mirror” effect without however being spangled or satin-like, or presenting “highlight points”.

This optical effect is measurable and quantifiable according to any method known to a person skilled in the art.

Typically, the optical effects are measurable and quantifiable by the method known to a person skilled in the art, the samba reflection method.

This method enables the specular integral and the Wvisual to be measured, which makes it possible to quantify and evaluate the visual appearance in terms of shine, colour and light scattering.

According to an embodiment, the cosmetic composition according to the invention has a specular integral greater than or equal to 6000 and a Wvisual greater than or equal to 8.

Preferably, the cosmetic composition according to the invention has a specular integral greater than or equal to 7000 and a Wvisual greater than or equal to 9.

A specular integral greater than or equal to 6000 and a Wvisual greater than or equal to 8 characterise the metallic effect sought in the context of the invention, in other words an aqueous metallic effect or “mirror effect”.

The metallic effect obtained according to the present invention differs from a shiny effect, a satin effect or a “highlight point” effect.

Typically, a specular integral approximately equal to 5000, typically between 4500 and 5500 and a Wvisual effect between 4 and 7, approximately 6, are characteristics of a shiny effect.

In the same manner, a specular integral less than 2500 with a Wvisual less than or equal to 3 are features of a satin effect.

### Additives

The composition according to the invention may comprise other ingredients as long as they do not interfere with the desired properties of the composition. These other ingredients may, for example, be preservatives, pH adjusters such as citric acid, sodium hydroxide or arginine, antimicrobial agents, perfumes, sun filters and the mixtures thereof.

### Preparation Method

The present invention also relates to a method for preparing a cosmetic composition according to the invention, comprising:

mixing the hydrophilic gelling agent with water, and optionally with the hydrocarbon and/or silicone emulsifiers, the humectants and the water-soluble solvent;
optionally adding film-forming agents, silicone and/or hydrocarbon emulsifiers and additives;
adding hydrophobic treated nacres and optionally hydrophobic treated pigments;
optionally adjusting the pH.

### Method for Making up Keratin Material

The present invention also relates to a method for making up keratin materials, in particular the skin or the lips, preferably the eyelids, consisting in applying on said keratin materials, in particular on the skin or the lips, preferably the eyelids, a cosmetic composition according to the invention.

In a preferred embodiment, the makeup method is a method for making up the eyelids.

### Cosmetic Use

The present invention also concerns the non-therapeutic cosmetic use of a composition according to the invention, in order to form, on keratin materials, in particular on the skin or the lips, preferably on the eyelids, a coloured makeup with aqueous metallic effect.

### EXAMPLE 1: EYE SHADOW

An eye shadow (OAP1) was prepared having the composition given in table 1 below:

TABLE 1

| INCI name | Content (% by weight) |
|---|---|
| Magnesium stearate coated nacres | 24.60 |
| CARBOMER | 0.32 |
| POLYSORBATE 20 | 0.24 |
| GLYCERIN | 3.00 |
| BUTYLENE GLYCOL | 3.00 |
| ALCOHOL | 5.00 |
| CAPRYLYL GLYCOL | 0.25 |
| CHLORPHENESIN | 0.27 |
| ISODECANE & | 12.00 including 5.34% |
| TRIMETHYLSILOXYSILICATE & | corresponding to the dry extract |
| AQUA & PROPANEDIOL & | of trimethylsiloxysilicate |
| DECYLGLUCOSIDE & | |
| PHENOXYETHANOL & | |
| CAPRYLYL GLYCOL & | |
| HEXYLENE GLYCOL (Granresin | |
| SIW-MQIZ) | |
| PEG-10 DIMETHICONE | 0.80 |
| AQUA | 50.13 |
| 25% sodium hydroxide solution | 0.34 |

The water, CARBOMER, POLYSORBATE 20, GLYCERIN, BUTYLENE GLYCOL and CAPRYLYL GLYCOL and CHLORPHENESIN were mixed while stirring in a propeller stirrer at 350 rpm.

The ISODECANE & TRIMETHYLSILOXYSILICATE & AQUA & PROPANEDIOL & DECYLGLUCOSIDE & PHENOXYETHANOL & CAPRYLYL GLYCOL & HEXYLENE GLYCOL and PEG-10 DIMETHICONE were then incorporated, while stirring in a propeller stirrer at 500 rpm.

The mixture obtained was then left to homogenise for 15 minutes.

The magnesium stearate coated nacres were then introduced, while still stirring at 500 rpm, until a smooth and a homogeneous texture was obtained.

Finally, the ALCOHOL was introduced and the mixture was allowed to homogenise for approximately 10 minutes.

The pH was adjusted using a sodium hydroxide solution.

### EXAMPLE 2: EYE SHADOW

An eye shadow (OAP2) was prepared having the composition given in table 2 below:

TABLE 2

| INCI name | Content (% by weight) |
|---|---|
| Magnesium stearate coated nacres | 24.60 |
| CARBOMER | 0.32 |
| POLYSORBATE 20 | 0.24 |
| GLYCERIN | 3.00 |
| BUTYLENE GLYCOL | 3.00 |
| ALCOHOL | 5.00 |
| PHENOXYETHANOL | 0.75 |
| ISODECANE & | 12.00 including 5.34% |
| TRIMETHYLSILOXYSILICATE & | corresponding to the dry extract |
| AQUA & PROPANEDIOL & | of trimethylsiloxysilicate |
| DECYLGLUCOSIDE & | |
| PHENOXYETHANOL & | |
| CAPRYLYL GLYCOL & | |
| HEXYLENE GLYCOL | |
| (Granresin SIW-MQIZ) | |
| PEG-10 DIMETHICONE | 0.80 |
| AQUA | 49.84 |
| 25% sodium hydroxide solution | 0.45 |

A premix of CARBOMER and water was produced. The POLYSORBATE 20, GLYCERIN, BUTYLENE GLYCOL, ALCOHOL and PHENOXYETHANOL were mixed into the premix while stirring.

The ISODECANE & TRIMETHYLSILOXYSILICATE & AQUA & PROPANEDIOL & DECYLGLUCOSIDE & PHENOXYETHANOL & CAPRYLYL GLYCOL & HEXYLENE GLYCOL and PEG-10 DIMETHICONE were then introduced into the previously obtained mixture.

The magnesium stearate coated nacres were then added.

The pH was adjusted using a sodium hydroxide solution.

### EXAMPLE 3: EYE SHADOW

In the following examples, 4 eye shadows, OAP3, OPA4, OAP5 and OAP6 respectively, were formulated by varying different parameters, such as:

the content of magnesium stearate coated nacres;
the treatment of the nacres or the absence of treatment;
the content of pigments and their treatment or non-treatment.

The different formulas are presented in the table below:

TABLE 3

| | Content (% by weight) | | | |
|---|---|---|---|---|
| INCI name | OAP 3 | OAP 4 | OAP 5 | OAP 6 |
| Magnesium stearate coated | 22.71 | — | — | 5.00 |
| nacres | | | | |
| Untreated nacres | — | 24.60 | 20.30 | — |
| Magnesium stearate coated | 1.87 | — | — | 19.58 |
| iron oxide | | | | |
| Untreated iron oxide | — | — | 4.35 | — |
| CARBOMER | 0.32 | 0.32 | 0.32 | 0.32 |
| POLYSORBATE 20 | 0.24 | 0.24 | 0.24 | 0.24 |
| GLYCERIN | 3.00 | 3.00 | 3.00 | 3.00 |
| BUTYLENE GLYCOL | 3.00 | 3.00 | 3.00 | 3.00 |
| ALCOHOL | 5.00 | 5.00 | 5.00 | 5.00 |
| PHENOXYETHANOL | 0.75 | 0.75 | 0.75 | 0.75 |
| ISODECANE & | 12.00 including | | | |
| TRIMETHYLSILOXYSILICATE & | 5.34% corresponding | | | |
| AQUA & PROPANEDIOL & | to the dry extract of | | | |
| DECYLGLUCOSIDE & | trimethylsiloxysilicate | | | |
| PHENOXYETHANOL & | | | | |
| CAPRYLYL GLYCOL & | | | | |
| HEXYLENE GLYCOL | | | | |
| (Granresin SIW-MQIZ) | | | | |
| PEG-10 DIMETHICONE | 0.80 | 0.80 | 0.80 | 0.80 |
| 25% sodium hydroxide solution | 0.34 | 0.45 | 0.45 | 0.38 |
| AQUA | 49.97 | 49.79 | 49.79 | 49.92 |

For OAP3, OAP4, OAP5, a premix of CARBOMER and water was produced. The POLYSORBATE 20, GLYCERIN, BUTYLENE GLYCOL, ALCOHOL and PHENOXYETHANOL were mixed into the premix while stirring.

The ISODECANE & TRIMETHYLSILOXYSILICATE & AQUA & PROPANEDIOL & DECYLGLUCOSIDE & PHENOXYETHANOL & CAPRYLYL GLYCOL & HEXYLENE GLYCOL and PEG-10 DIMETHICONE were then introduced into the previously obtained mixture.

The following were then added:

magnesium stearate coated nacres and magnesium stearate coated iron oxide for OAP3;
untreated nacres for OAP4;
untreated nacres and untreated iron oxide for OAP5.

The pH was adjusted using a sodium hydroxide solution.

For OAP6, a premix of CARBOMER, GLYCERIN, BUTYLENE GLYCOL and water was prepared.

The POLYSORBATE 20, ALCOHOL and PHENOXYETHANOL were then mixed into the premix while stirring.

The ISODECANE & TRIMETHYLSILOXYSILICATE & AQUA & PROPANEDIOL & DECYLGLUCOSIDE & PHENOXYETHANOL & CAPRYLYL GLYCOL & HEXYLENE GLYCOL and PEG-10 DIMETHICONE were then introduced into the previously obtained mixture.

Finally, the magnesium stearate coated nacres and magnesium stearate coated iron oxide where introduced.

The pH was adjusted using a sodium hydroxide solution.

### EXAMPLE 4: EVALUATION OF THE METALLIC EFFECT

### Measurement and Quantification by Samba® Reflection

The shine of the formulas was evaluated by means of a Samba® apparatus marketed by BOSSA NOVA TECHNOLOGIES (Venice, USA), a method particularly known to a person skilled in the art in the field of cosmetics for evaluating shine.

The Samba® system is based on the use of a polarised camera allowing the visual appearance to be evaluated in terms of shine, colour and scattering of the light on different media. It involves a contact free measurement allowing measurements to be carried out immediately after application of products (without drying).

The system is composed of three main elements:

a colour polarisation camera
a polarised illumination
a cylindrical support.

The scene observe by the polarisation camera corresponds to the application card and the product placed on the cylindrical mounting part, with −12.7 mm<x<12.7mm and −45°<theta<45°. This system is equivalent to the goniometric measurement thanks to the cylindrical support.

The polarisation of the colour camera and the illumination make it possible to break down the scattered light into specular light and diffuse light for each pixel of the image. The colour analysis allows a breakdown of the specular light into the shine and chrominance components.

The source sends a polarised light beam onto the sample. When this beam comes into contact with the surface, it is returned to the camera in a plurality of light components:

The first component is the shine. This is a white polarised reflection from the surface, which has the same polarisation as the incident light. It appears as a band on the product. The width of the band is determined by the roughness of the surface and the irregularities of the product and/or the application.
The second component is called the chrominance band (chroma).
The third component is the diffused light.

It is possible to visualise and record the different images (sum, specular, shine, chroma and diffuse) and the corresponding angular spectral profiles.

The numerical data are calculated, allowing a complete characterisation of the visual appearance of the product. The measurements are carried out on the image or on the curve.

Typically, the larger the reflectance peak (specular light), the shinier the product.

### Calculation of the Specular Integral and the Wvisual

The first step consists of acquiring images of the sample.

The second step consists of extracting angular distributions and collecting:

the specular profile;
the diffuse profile;
and the geometry of the shine band.

These profiles are characterised in a third step.

The specular profile makes it possible to obtain the following information:

the maximum of the specular profile;
the specular integral
the width at half height of the band.

The diffused profile makes it possible to obtain the following observations:

the maximum of the diffusion profile;
the diffused integral.

The Wvisual in degrees is calculated in the following manner:

the image is divided into 10 then, on each portion of the image, the reflection curve is traced and the width at half height of the peak is calculated. An average is then performed over the 10 curves.

Wvisual (deg)=width at half height of the peak at Max/2 of the reflectance peak (specular light) measured on the image, [Math 1]

Max: maximum the profile
Integral: area under the reflectance curve (specular light).

Typically, the smaller Wvisual, the shinier the product.

The following rules have been used to perform the evaluations of shine of the formulas on the Samba® apparatus marketed by BOSSA NOVA TECHNOLOGIES (Venice, USA):

application to the pneumatic applicator;
deposit of 150 μm;
measurement at T0 at room temperature T approximately 20-22° C.;
application on Black Contrast Cards from BYK (ref. 2810).

### Evaluation of the Aqueous Metallic Effect of Different Eye Shadows

The different eye shadows have been evaluated using the Samba® method as described above in order to characterise the metallic effect according to the present invention.

The presence or absence of magnesium stearate coated nacres as well as the presence or absence of stearate coated iron oxide is summarised in the table below:

TABLE 4

| | Nacres/Pigments | | | |
|---|---|---|---|---|
| | Treated | Untreated | Treatediron | Untreated iron |
| | nacres | nacres | oxide | oxide |
| | Content (% by weight) | | | |
| OAP1 | 24.60 | — | — | — |
| OAP2 | 24.60 | — | — | — |
| OAP3 | 22.71 | — | 1.87 | — |
| OAP4 | — | 24.60 | — | — |
| OAP5 | — | 20.30 | — | 4.35 |
| OAP6 | 5.00 | — | 19.60 | — |

The Wvisual measured on the image, the Maximum, the integral measured on the curve and the effect observed with the naked eye are recorded in the table below for each of the eye shadows.

TABLE 5

| | Specular | | | |
|---|---|---|---|---|
| | Wvisual (deg) | | Integral | Effect observed |
| | measured on | | measured | with the |
| | the image | Max | on the curve | naked eye |
| OAP1 | 9 | 260.48 | 6998 | aqueous metallic effect |
| OAP2 | 11 | 333.78 | 7253 | aqueous metallic effect |
| OAP3 | 6 | 140.47 | 5165.67 | satin metallic effect |
| OAP4 | 6 | 212.28 | 4757 | satin metallic effect |
| OAP5 | 3 | 257.63 | 1582 | satin effect |
| OAP6 | 3 | 135.61 | 2272 | satin effect |

Advantageously, the cosmetic compositions according to the present invention and comprising hydrophobic treated nacres make it possible to obtain the desired metallic effect, called aqueous metallic effect or mirror effect.

### EXAMPLE 5: EYE SHADOW

An eye shadow (OAP7) was prepared having the composition given in table 6 below:

TABLE 6

| INCI name | Content (% by weight) |
|---|---|
| Magnesium stearate coated nacres | 24.66 |
| CARBOMER | 0.32 |
| POLYSORBATE 20 | 0.24 |
| GLYCERIN | 3.00 |
| BUTYLENE GLYCOL | 3.00 |
| ALCOHOL | 5.00 |
| PHENOXYETHANOL | 0.75 |
| ISODECANE & | 12.00 including |
| TRIMETHYLSILOXYSILICATE & | 5.34% corresponding |
| AQUA & PROPANEDIOL & | to the dry extract of |
| DECYLGLUCOSIDE & | trimethylsiloxysilicate |
| PHENOXYETHANOL & | |
| CAPRYLYL GLYCOL & | |
| HEXYLENE GLYCOL (Granresin | |
| SIW-MQIZ) | |
| PEG-10 DIMETHICONE | 0.80 |
| AQUA | 49.90 |
| 25% sodium hydroxide solution | 0.34 |

A premix of CARBOMER and water was produced. The POLYSORBATE 20, GLYCERIN, BUTYLENE GLYCOL, ALCOHOL and PHENOXYETHANOL were mixed into the premix while stirring.

The ISODECANE & TRIMETHYLSILOXYSILICATE & AQUA & PROPANEDIOL & DECYLGLUCOSIDE & PHENOXYETHANOL & CAPRYLYL GLYCOL & HEXYLENE GLYCOL and PEG-10 DIMETHICONE were then introduced into the previously obtained mixture.

The magnesium stearate coated nacres were then added.

The pH was adjusted using a sodium hydroxide solution.

Once obtained, the eye shadow OAP7 was tested in order to evaluate the retention of the metallic effect obtained. The evaluations were carried out by an experimenter:

immediately after application of the composition on the eyelids;
before the meal at time 3 hours 30 minutes;
at the end of the day at time 7 hours 30 minutes.

Scores between 0 and 9 were assigned by the experimenter for:

the presence of the product;
the homogeneity of the eye shadow;
migration of the eye shadow or diffusion of the eye shadow into the crease of the eyelid;
diffusion of the eye shadow around the eye.

These scores are used to calculate:

the average at the three times;
the delta—Δ—for each time (3 hours 30 minutes, 7 hours 30 minutes).

The deltas are then expressed as a percentage (%):

% loss of product (tint retention);
% loss of homogeneity;
% diffusion of the product around the eye;
% migration.

The results are recorded in table 7 below:

TABLE 7

| | | | Retention at 3 | |
|---|---|---|---|---|
| | | Makeup | hours 30 | Retention at 7 |
| | Application | result | minutes | hours 30 |
| Comments | The ease of | Intense; | Fades very | Does not fade |
| | working; | Homogeneous; | slightly; | further; |
| | Rather simple | Does not | A little less | No further loss |
| | to make | diffuse under | homogeneous; | of homogeneity; |
| | homogeneous. | the eye. | Slight | Slight migration; |
| | | | migration; | Does not diffuse |
| | | | Does not | under the eye. |
| | | | diffuse under | |
| | | | the eye. | |

In general, OAP7 according to the invention has a good working time, allows a homogeneous application of makeup and an intense result. The eye shadow fades very slightly at the end of 3 hours 30 minutes, losing homogeneity very slightly, but hardly migrates at all and does not diffuse under the eye.

Thus and advantageously, the cosmetic compositions in a continuous aqueous phase according to the present invention, comprising hydrophobic treated nacres make it possible to obtain the desired metallic effect, called aqueous metallic effect or mirror effect, and have a long-retention, fresh and comfortable texture.

1. A cosmetic composition comprising, in a continuous aqueous phase,
at least one colouring agent chosen from hydrophobic treated nacres;
at least one hydrophilic gelling agent; and
water.

2. The cosmetic composition according to claim 1, wherein the hydrophobic treated nacres are present with a content ranging from 16 to 35% by weight, preferably from 20 to 33% by weight, with respect to the total weight of the composition.

3. The cosmetic composition according to claim 1, wherein the hydrophobic treatment is a treatment by metallic soap.

4. The cosmetic composition according to claim 1, wherein the metallic soap is a fatty acid soap having 12 to 22 carbon atoms, and in particular 12 to 18 carbon atoms.

5. The cosmetic composition according to claim 1, wherein the metal of the metallic soap is zinc or magnesium.

6. The cosmetic composition according to claim 1, wherein the metallic soap is chosen from zinc laurate, magnesium stearate, magnesium myristate, zinc stearate and the mixtures thereof, and the metallic soap is preferably magnesium stearate.

7. The cosmetic composition according to claim 1, wherein the hydrophilic gelling agent is chosen from the polysaccharides such as the exudates of microorganisms, preferably xanthan gum, plant extracts, preferably tremella fuciformis polysaccharide, acrylic acid polymers, preferably carbomer, or one of the mixtures thereof.

8. The cosmetic composition according to claim 1, wherein the hydrophilic gelling agent is present with a content ranging from 0.05 to 3% by weight, preferably from 0.1 to 2% by weight, with respect to the total weight of the composition.

9. The cosmetic composition according to claim 1, comprising 30 to 70% by weight water, preferably 40 to 60% by weight, with respect to the total weight of the composition.

10. The cosmetic composition according to claim 1, wherein the aqueous phase comprises at least one other colouring agent chosen from the hydrophobic treated pigments, the content of hydrophobic treated pigments with respect to the total weight of the composition being less than 1.5%.

11. The cosmetic composition according to claim 1, further comprising a humectant, chosen from the polyols and preferably chosen from glycerol and butylene glycol.

12. The cosmetic composition according to claim 1, further comprising a film-forming agent chosen from a film-forming polymer dispersible in the aqueous phase, preferably an aqueous dispersion of trimethylsiloxysilicate resins, a liposoluble film-forming polymer such as trimethylsiloxysilicate resins dispersed in isododecane, acrylate/polytrimethylsiloxymethacrylate copolymers comprising a carbosiloxane dendrimer structure grafted on a vinyl backbone and the mixtures thereof.

13. The cosmetic composition according to claim 1, further comprising at least one oil and at least one emulsifier, said composition being in the form of an oil-in-water emulsion.

14. The cosmetic composition according to claim 13, wherein the oil is a volatile oil chosen from volatile hydrocarbon oils, volatile linear alkanes and volatile silicon oils.

15. The cosmetic composition according to claim 13, wherein the composition comprises less than 5% non-volatile oils and, preferably, the composition is free of non-volatile oils.

16. The cosmetic composition according to claim 13, wherein the emulsifier is chosen from silicone emulsifiers, hydrocarbon emulsifiers or the mixtures thereof.

17. The cosmetic composition according to claim 1, wherein the cosmetic composition has a specular integral greater than or equal to 6000, preferably greater than or equal to 7000 and a Wvisual greater than or equal to 8, preferably greater than or equal to 9.

18. A method for preparing a cosmetic composition according to claim 1, comprising:
mixing the hydrophilic gelling agent with water, and optionally with the hydrocarbon and/or silicone emulsifiers, the humectants and the water-soluble solvents;
optionally adding film-forming agents, silicone and/or hydrocarbon emulsifiers and additives;
adding hydrophobic treated nacres and optionally hydrophobic treated pigments; and
optionally adjusting the pH.

19. A method for making up keratin materials, in particular the skin or the lips, preferably the eyelids, comprising applying on said keratin materials, in particular the skin or the lips, preferably the eyelids, a cosmetic composition according to claim 1.

20. A cosmetic composition according to any one of claim 1, for application on keratin materials, in particular on the skin or the lips, preferably on the eyelids, a coloured makeup with metallic effect.
